(19) United States
(12) Patent Application Publication
(10) Pub. No.: US 2007/0129547 A1
(43) Pub. Date: Jun. 7, 2007

(54) Process to prepare substituted imidazopyrazine compounds

(76) Inventors: Kristen Michelle Mulvihill, Farmingdale, NY (US); Arlindo L. Castelhano, Farmingdale, NY (US)

Correspondence Address: OSI PHARMACEUTICALS, INC., 41 PINELAWN ROAD, MELVILLE, NY, 11747, US

(21) Appl. No.: 11/635,291

(22) Filed: Dec. 7, 2006

Related U.S. Application Data
(60) Provisional application No. 60/748,120, filed on Dec. 7, 2005.

(51) Int. Cl. A61K 31/498; C07D 487/04

(52) U.S. Cl. 544/350

(57) ABSTRACT

A method of preparing 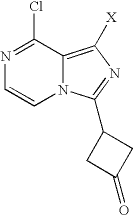
wherein, X=Cl, Br, I, comprises the step of treating 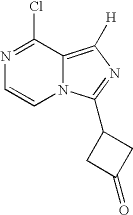 with N-chloro-, N-bromo-, or N-iodosuccinimide in a compatible solvent such as dimethylformamide (DMF) at 0-60° C. followed by halogenation.

This application claims the benefit of U.S. Patent Application No. 60/748,120 filed Dec. 7, 2005.

### BACKGROUND OF THE INVENTION

The present invention is directed to a process of preparing substituted imidazopyrazines. In particular, the present invention is directed to a process of preparing cyclobutane substituted imidazopyrazines.

Substituted imidazopyrazine compounds are useful in the treatment of diseases, including cancer, as disclosed in U.S. Patent Publication No. US2004 0186124A (International Patent Publication No. PCT/US2005/010606), and International Patent Publication No. WO 2005/037836. It is desirable to develop novel processes to prepare central intermediates for elaboration into more complex drug molecules. There is a need to make these materials in practical, versatile, and potentially inexpensive methods.

### SUMMARY OF THE INVENTION

A method of preparing 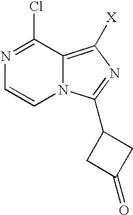
wherein, X=Cl, Br, I, comprises the step of treating 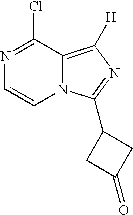
with N-chloro-, N-bromo-, or N-iodosuccinimide in a compatible solvent such as dimethylformamide (DMF) at 0-60° C. followed by halogenation.

### DETAILED DESCRIPTION OF THE INVENTION

In general, referring to Scheme 1, 3-substituted-8-chloroimidazopyrazines can be assembled by the condensation of 2-aminomethyl-3-chloropyrazine 1 with an activated aryl, heteroaryl, alkyl, or cycloalkyl carboxylic acids, 2. The latter can be activated as active esters, acid chlorides, or coupled with amine 1 using established amide coupling agents such as DCC (N,N′-Dicyclohexylcarbodiimide), CDI (1,1′-Carbonyldiimidazole), chloroformates such as isobutyl chloroformate, or phosphorous-based amide coupling agents such as phenyl N-phenylphosphoramidochloridate or diphenylphosphinic chloride, or other amide forming agents known in the art. The amide 2 is subsequently treated with a dehydrating agent such as Vilsmeier reagent to undergo a dehydration-cyclization reaction to give imidazopyrazine 3. The latter can be halogenated cleanly at the C-1 position with NCS (N-chlorosuccinimide), NBS (N-bromosuccinimide) or NIS (N-iodosuccinimide) to give a highly functionalized imidazopyrazine system 4 (X=Cl, Br, I), ready for further elaboration at the C-1 and C-8 positions in the core molecule and the appendage at C-3. 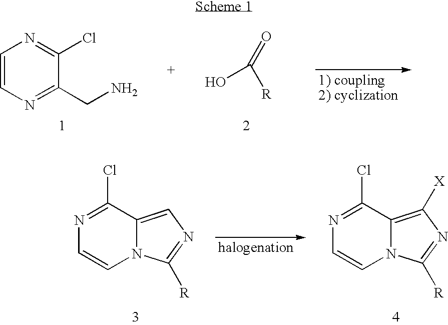
wherein, X is Cl, Br, I; and R is aryl, heteroaryl, biaryl, arylalkyl, alkyl, cycloalkyl, bicyclic, each substituted with 1-3 substituents.

The carboxylic acid partner 2 in the amide condensation reaction is commercially available or in the case of 2 wherein R is 3-oxocyclobutyl, the compound can be made following a literature procedure described by Pigou, P. E.; Schiesser, C. H. J. Org. Chem. 1988, 53, 3841-3843.

The starting pyrazine amine 1 is reported to be available through commercial supply houses. Herein, amine 1 is prepared by the conversion of the corresponding 2-hydroxymethyl or 2-chloromethyl-3-chloropyrazine through the intermediacy of the corresponding phthalamido, diformylamide, or hexamethyltetramnine adducts followed by hydrazinolysis or acid hydrolysis (see Scheme 2). Amine 1 is conveniently isolated as a hydrochloride salt. The 2-hydroxymethyl-3-chloropyrazine precursor, 7a, is prepared from the reaction of 2-chloropyrazine lithio anion with DMF or other formylating agents followed by in-situ reduction with NaBH₄. Treatment of the latter alcohol with SOCl₂ generates 2-chloro-3-chloromethylpyrazine 7b, a known compound described in U.S. Pat. No. 3,625,944). Reaction of 7b with hexamethylenetetramine, sodium diformylamide, or potassium phthalamide generates 8a-c. Other aminating agents such as sodium azide or sodium hexamethyldisilazide can also be used. The phthalamide precursor of amine 1 is also made from 2-hydroxymethyl-3-chloropyrazine using Mitsunobu conditions (JS2004 0186124A).

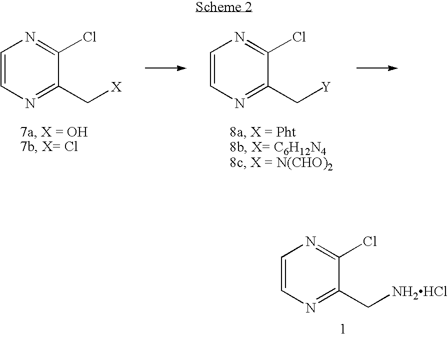

Compounds described herein contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The present invention includes all stereoisomers of 3 and 4 and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium slats. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylameine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric and tartaric acids.

In an aspect of the invention, a method of preparing 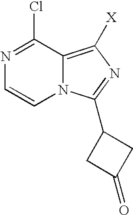
wherein, X=Cl, Br, I, comprises the step of treating 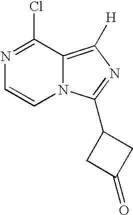
with N-chloro-, N-bromo-, or N-iodosuccinimide in a compatible solvent followed by halogenation.

The solvent may be, for example, ethyl acetate (EtOAc), acetonitrile (CH₃CN), or dimethylformamide DMF).

The treating step may be performed at at emperature of from about 0-60° C.

In another aspect of the invention, a method of preparing 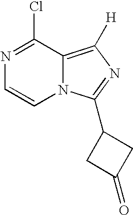
comprises treating 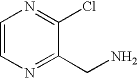
with an acid chloride or activated carboxylic acid to form the corresponding amide; and then inducing a dehydration-cylization in a solvent with Vilsmeier's reagent prepared in situ with phosphoryl chloride (POCl₃) and DMF under anhydrous conditions.

The solvent used can be, for example, ethyl acetate (EtOAc), acetonitrile (CH₃CN), or DMF.
Experimental Details:

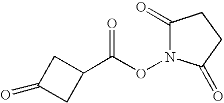

3-Oxo-cyclobutanecarboxylic acid 2,5-dioxo-pyrrolidin-1-yl ester: N-Hydroxysuccinimide (202 mg, 1.75 mmol), N,N′-dicyclohexylcarbodiimide (362 mg, 1.75 mmol), and 3-oxo-cyclobutanecarboxylic acid (200 mg, 1.75 mmol) (Pigou, P. E.; Schiesser, C. H. J., Org. Chem., 1988, 53, 3841-3843) were suspended in isopropyl acetate (5.1 mL) and the reaction was stirred at rt. After 1 h, the reaction was filtered to remove the urea byproduct. The filtrate was concentrated in vacuo to provide the title compound as a white solid. ¹H NMR (400 MHz, DMSO-d₆) δ 2.83 (bs, 4H), 3.30-3.39 (m, 2H), 3.52-3.60 (m, 2H), 3.67-3.73 (m, 1H).

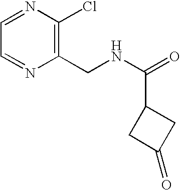

3-Oxo-cyclobutanecarboxylic acid (3-chloro-pyrazin-2-ylmethyl)-amide: 3-Oxo-cyclobutanecarboxylic acid 2,5-dioxo-pyrrolidin-1-yl ester (284 mg, 1.35 mmol), C-(3-chloro-pyrazin-2-yl)-methylamine hydrochloride salt (243 mg, 1.35 mmol), and NaHCO₃ (298 mg, 3.55 mmol) were dissolved in THF (2.0 mL) and water (2.0 mL) and the reaction was stirred at rt. After 30 min, the layers were allowed to separate and the aqueous layer was removed. The aqueous layer was back extracted with EtOAc. The organics were dried over MgSO₄, filtered, and concentrated in vacuo to provide the title compound as a waxy pale yellow solid. ¹H NMR (400 MHz, DMSO-d₆) δ 3.10-3.27 (m, 5H), 4.56 (d, 2H, J=5.6 Hz), 8.44 (d, 1H, J=3.2 Hz), 8.63 (d, 1H, J=2.4 Hz), 8.73 (t, 1H, J=5.2 Hz).

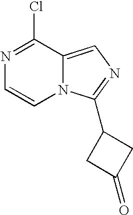

3-(8-Chloro-imidazo[1,5-a]pyrazin-3-yl)-cyclobutanone: 3-Oxo-cyclobutanecarboxylic acid (3-chloro-pyrazin-2-ylmethyl)-amide (18.60 g, 0.0776 mol) was suspended in ethyl acetate (167 mL). DMF (7.51 mL, 0.097 mol) was added followed by POCl₃ (9.04 mL, 0.097 mol). After 2 h, the reaction was poured into saturated Na₂CO₃ (54 mL). The organic layer was removed and the aqueous layer was back-extracted with EtOAc (1×55 mL, 2×100 mL). The combined organics were concentrated in vacuo to provide the title compound as a tan solid. ¹H NMR (400 MHz, CDCl₃) δ 3.59-3.68 (m, 2H), 3.72-3.79 (m, 2H), 3.86-3.94 (m, 1H), 7.40 (d, 1H, J=5.2 Hz), 7.60 (d, 1H, J=5.2 Hz), 7.85 (s, 1H).

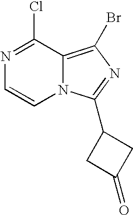

3-(1-Bromo-8-chloro-imidazo[1,5-a]pyrazin-3-yl)-cyclobutanone: N-Bromosuccinimide (1.67 g, 9.38 mmol) was dissolved in DMF (6 mL) under an atmosphere of nitrogen. 3-(8-Chloro-imidazo[1,5-a]pyrazin-3-yl)-cyclobutanone (2.02 g, 8.44 mmol) was dissolved in DMF (6 mL) and added to the reaction. After 5 min, the dark brown reaction was poured into brine (50 mL) and the aqueous solution was extracted with EtOAc (2×50 mL). The combined organics were concentrated in vacuo to a brownish solid. The crude material was recrystallized from isopropanol and water (10:1) to provide the title compound as tan needles. ¹H NMR (DMSO-d₆, 400 MHz): δ 3.45-3.53 (m, 2H), 3.58-3.67 (m, 2H), 4.08-4.16 (m, 1H), 7.45 (d, 1H, J=5.2 Hz), 8.30 (d, 1H, J=4.8 Hz).

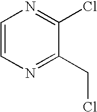

2-Chloro-3-chloromethyl-pyrazine: (3-Chloropyrazin-2-yl)-methanol (8.00 g, 0.0553 mol) was dissolved in toluene (40 mL) and N,N-dimethylformamide (2.1 mL, 0.028 mol). The solution was treated dropwise with thionyl chloride (4.44 mL, 0.0609 mol) over 10 min, keeping the temperature at 40° C. during the addition. After 30 min the reaction was quenched with 20 mL water and then solid sodium carbonate (7.7 g, 0.073 mol) was added to give a neutral pH. The mixture was filtered and the toluene layer was separated. The aqueous layer was re-extracted with 25 mL toluene and the combined toluene extracts were washed with water (25 mL). The toluene solution was evaporated to remove water. The product can be used without further purification or if desired purified by distillation at 58° C. (1 Torr) to give the title compound a colorless liquid. ¹H NMR (CDCl₃, 400 MHz): δ 4.82 (s, 2H), 8.32-8.43 (m, 1H), 8.47-8.57 (m, 1H). m/z [MH⁺] 163/165/167 (100/60/10).

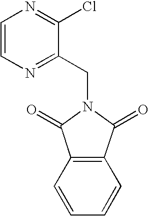

2-(3-Chloro-pyrazin-2-ylmethyl)-isoindole-1,3-dione: 2-Chloro-3-chloromethylpyrazine (1.0 g, 6.1 mmol) was added to a suspension of potassium phthalimide (2.3 g, 12.2 mmol) in dimethylformamide (10 mL). The mixture was heated at 60° C. for 17 h. The resultant suspension was evaporated to dryness and the residue was suspended in water (5 mL). The 2-(3-chloro-pyrazin-2-ylmethyl)-isoindole-1,3-dione was collected by filtration and was washed with water. The solid was dried to yield the title compound. ¹H NMR (CDCl₃, 400 MHz): δ 5.15 (s, 2H), 7.72-7.80 (m, 2H), 7.88-7.95 (m, 2H), 8.25-8.29 (m, 1H), 8.29-8.34 (m, 1H). m/z [MH⁺] 274/276 (100/35).

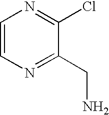

C-(3-Chloro-pyrazin-2-yl)-methylamine hydrochloride (from 8a): To a suspension of 2-(3-chloro-pyrazin-2-ylmethyl)-iso indole-1,3-dione (90.0 g, 0.329 mol) in methanol (1800 mL) was added hydrazine (20.6 mL, 0.658 mol). After 3 min the mixture formed a thick white suspension, the mixture was gently heated to 60° C. A clear yellow solution formed which turned cloudy after 15 min. After 1 h the reaction was complete. The reaction mixture was cooled to 20° C. and filtered. The bulk of the fibrous residue was slurried in 6×450 mL toluene and this was also filtered. The combined filtrates were concentrated by evaporation to approx. 1.5 L. The resultant slurry was filtered and the cake was washed with 900 ml toluene. The combined solution was concentrated to 1 L, filtered and diluted to a total volume of 1.35 L. The solution was re-filtered and then hydrogen chloride gas was added to the rapidly stirring mixture. A cream colored solid which was collected by filtration and was washed with 100 ml toluene. The solid was dried under vacuum to give the title compound as the hydrochloride salt. ¹H NMR (CD₃OD, 400 MHz): δ 4.47 (s, 2H), 8.38-8.54 (m, 1H), 8.56-8.72 (m, 1H).

C-(3-Chloro-pyrazin-2-yl)-methylamine hydrochloride (from 8b): 1-(3-Chloro-pyrazin-2-ylmethyl)-3,5,7-triaza-1-azonia-tricyclo[3.3.1.1*3,7*]decane; chloride (6.0 g, 0.022 mol) was suspended in methanol and 37% hydrochloric acid (5.0 mL, 0.06 mol) was added. The mixture was heated to reflux to give a clear orange solution. After 5 min solid began to precipitate. After 1 h the heating was stopped and the reaction mixture was treated with sodium carbonate (3.2 g, 0.030 mol) and 10 mL of water. Toluene 50 mL was added and the mixture was concentrated by evaporation to remove water and methanol. A further 2×50 mL toluene was added and evaporated. Then IPA (50 mL) was added which caused salts to precipitate. The mixture was filtered and concentrated by evaporation followed by addition of further IPA (50 mL), it was again filtered to give a clear orange brown solution. The solution was treated with HCl gas until absorption has stopped. The resultant suspension was stirred for 30 minutes and the solid was collected by filtration. The cake was washed with IPA and the solid was dried under vacuum to give the title compound.

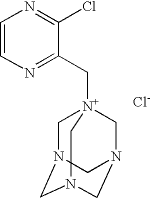

1-(3-Chloro-pyrazin-2-ylmethyl)-3,5,7-triaza-1-azonia-tricyclo[3.3.1.1*3,7*]decane; chloride: 2-Chloro-3-chloromethylpyrazine (1.00 g, 0.00613 mol) and hexamethylenetetramine (1.1 g, 0.0076 mol) in chloroform (5 mL) was heated to reflux. After 9 h the slurry was allowed to cool and the solid was collected by filtration. The cake was washed with 3×1 mL chloroform and the solid was dried under vacuum at room temperature to yield the title compound. ¹H NMR (d6-DMSO, 400 MHz): δ 4.31 (s, 2H), 4.40-4.68 (m, 6H), 5.35 (s, 6H), 8.71 (d, 1H), 8.83 (d, 1H). m/z [M⁺] 267/269.

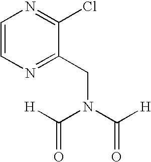

N-(3-Chloro-pyrazin-2-ylmethyl)-N-formyl-formamide: 2-Chloro-3-chloromethylpyrazine (1.00 g, 0.00613 mol) and diformylamide sodium salt (0.87 g, 0.0092 mol) in N,N-dimethylformamide (10 mL) was stirred at rt. After 8 h the solvent was removed by evaporation and water (10 mL) was added to the residue. The mixture was extracted with 3×5 mL ethyl acetate and the combined extracts were washed with 10 mL water. The organic solution was dried over sodium sulfate, filtered and the solvent was evaporated to yield the title compound as a brown oil which solidified on standing under high vacuum. ¹H NMR (CDCl₃, 400 MHz): δ 5.10 (s, 2H), 8.21-8.43 (m, 2H), 9.08 (s, 2H). m/z [MH⁺] 200/202 (35/10), 172/174 (100/35).

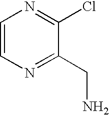

C-(3-Chloro-pyrazin-2-yl)-methylamine (from 8c): A suspension of N-(3-Chloro-pyrazin-2-ylmethyl)-N-formyl-formamide (0.400 g, 0.002 mol) in isopropyl alcohol (4 mL) was treated with 37% hydrogen chloride (0.182 mL, 0.0022 mol) and was heated to reflux. After 1 h 13% of an intermediate (probably the formamide) remained. 37% Hydrogen chloride (33 μL, 0.00040 mol) was added and the reflux continued. After 1 h 3% of the intermediate remained. 37% Hydrogen chloride (6 μL, 0.0002 mol) was charged and the heating was continued for a further 1 h. The solvent was then removed by evaporation and the pale brown solid was dried under high vacuum to yield the title compound as the hydrochloride salt.

What is claimed is:

1. A method of preparing 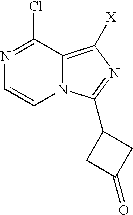
wherein, X=Cl, Br, I, comprising:
treating 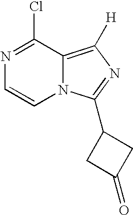
with N-chloro-, N-bromo-, or N-iodosuccinimide in a compatible solvent followed by halogenation.

2. The method of claim 1, wherein the treating step is performed in ethyl acetate, acetonitrile, or dimethylformamide.

3. The method of claim 2, wherein the treating step is performed at 0-60° C.

4. A method of preparing 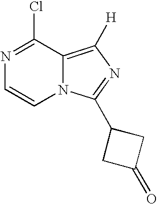
comprising:
treating 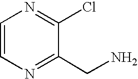
with an acid chloride or activated carboxylic acid to form the corresponding amide; and
inducing a dehydration-cylization in a solvent with Vilsmeier's reagent prepared in situ with phosphoryl chloride (POCl₃) and dirnethylformamide under anhydrous conditions.

5. The method of claim 3 wherein the solvent is ethyl acetate, acetonitrile, or dimethylformamide.
